(19) United States
(12) Patent Application Publication
(10) Pub. No.: US 2004/0121949 A1
(43) Pub. Date: Jun. 24, 2004

(54) Composition comprising partly-hydrolysed fish gelatin and a method for treatment of a patient suffering from arthrosis, osteoporosis and other diseases

(76) Inventors: Michele Bonanomi, Roma (IT); Mauro De Gregorio, Roma (IT)

Correspondence Address: PEARNE & GORDON LLP, 1801 EAST 9TH STREET, SUITE 1200, CLEVELAND, OH, 44114-3108, US

(21) Appl. No.: 10/324,857

(22) Filed: Dec. 20, 2002

(51) Int. Cl. A61K 38/38

(52) U.S. Cl. 514/12; 530/354

(57) ABSTRACT

The present invention relates to a composition comprising partly-hydrolysed fish gelatin and a method for treatment of a patient suffering from arthrosis, osteoporosis and other diseases.

Furthermore, the present invention relates to use of partly-hydrolysed fish gelatin as a medical speciality. In particular, the present invention refers to use of partly-hydrolysed fish gelatin for preparation of food additives or supplements in the medical field, as a source of easily assimilable amino acids useful for trophism of the osteo-articular system, scalp, nails and other particular tissues, in addition to being useful for the whole organism during senescence, pregnancy, convalescence and other conditions sharing a deficient supply, an increased consumption or a defective absorption of amino acids, both in the human and animal field.

### BACKGROUND OF THE INVENTION

1. Field of the Invention

The present invention relates to a composition comprising partly-hydrolysed fish gelatin and a method for treatment of a patient suffering from arthrosis, osteoporosis and other diseases. Furthermore, the present invention relates to use of a partly-hydrolysed fish gelatin as a medical speciality. In particular, the present invention relates to use of partly-hydrolysed fish gelatin for preparation .of food additives to use as supplements in the medical field, as a source of easily assimilable amino acids useful for trophism of the osteo-articular system, scalp, nails and other particular tissues, in addition to being useful for the whole organism during senescence, pregnancy, convalescence and other conditions that all have in common a deficient supply, an increased consumption or a defective absorption of amino acids, both in the human and animal field.

2. Prior art

It is known that gelatin obtained from connectivum contains essential and nonessential amino acids in proportions corresponding to those of the bone, the articulations, the keratin and other connective tissues. Oral administration of same to men and laboratory animals, in amounts at least as high as 100 mg/kg, showed a favourable effect under various situations and conditions. By way of example, oral administration of same to an amount of 10 g/day has a well-documented effect on pain and motility in patients suffering from arthrosis, osteoporosis and other diseases of the locomotor apparatus (Adam M., Therapiewoche (1991) 41 2456-61).

In an amount of 100 mg/kg/day it improves hair trophism both in men and laboratory animals (Silvestrini B., “Method for increasing hair growth” U.S. Pat. No. 4,749,684 (1988); Scala J., Hollies N. R. S., Sucher K. P., Nutr. Report Inter. (1976) 13 579-592).

Oral administration of gelatin to the above mentioned dosages is not easy and it is often badly tolerated because this substance in the stomach swells giving rise to an unpleasant feeling of repletion. In addition, in some persons it may cause reaction of the allergic type.

To avoid the above mentioned drawbacks, resorting to a completely hydrolysed gelatin improving amino acid absorption was proposed in the past, so as to enable dosage halving and avoid gastric trouble (Silvestrini B., and Kirschner G., “Integratori alimentari e dietetici contenenti gelatina idrolizzata e loro uso in campo medico” Patent IT 01299131).

However, the completely hydrolysed gelatin too has some drawbacks. In patent IT 01299131 the gelatin hydrolysis is obtained by chemical route or, alternatively, by enzymatic route.

It is known that gelatin hydrolysis obtained by chemical route, is generally carried out by heating with concentrated hydrochloric acid to a temperature of about 110° C. for a time included between 18 and 24 hours. This operating modality appears of difficult application on a preparation scale. In addition, recent bibliographic references (Fountoulakis M., Lahm H. W., J. Chromatogr. A, (1998) 826 109-134) clearly highlight formation of decomposition products the nature of which is a function of the employed experimental conditions.

It is known that gelatin hydrolysis, obtained by enzymatic route is surely a less drastic process, but it has drawbacks as well.

A first drawback is connected with the presence of free sulfurised amino acids derived from hydrolytic processes. Owing to the presence of sulfurised amino acids the organoleptic properties of the gelatin are worsened, due to sulphur smell and aftertaste that cannot be accepted by all consumers and patients.

Alternatively, mixtures could be used that consist of the individual amino acids in the proportions found in collagen.

The last-mentioned solution too is not devoid of shortcomings. A first shortcoming refers to the related high costs for accomplishment of these amino acid mixtures. A high cost is hardly compatible with the features of a treatment involving administrations over long periods of time, in relatively high amounts.

In addition, the presence of potentially toxic impurities is not to be excluded, which impurities result from both extractive and synthetic processes employed for obtaining the individual amino acids constituting the mixture.

### SUMMARY OF THE INVENTION

The technical problem to be solved consists in making a source of amino acids more bioavailable for the metabolism of the human and animal organism, said amino acids being easily assimilable and coming from a gelatin that does not show the drawbacks of the known art.

In particular, the technical problem to be solved consists in making a source of amino acids less bioavailable for the metabolism of the human and animal organism, so that diseases arising from a lack of amino acids in the living organism can be treated while avoiding the drawbacks of the known art.

The solution to this problem is proposed by the Applicant that found it useful to employ a partly-hydrolysed fish gelatin.

It is a first object of the present invention to provide a partly-hydrolysed fish gelatin for uses as a food additive or supplement having the features set out in the appended independent claim.

It is another object of the present invention to provide a composition comprising a partly-hydrolysed fish gelatin for use as a food additive or supplement and/or medical speciality, having the features set out in the appended independent claim.

It is a further object of the present invention to provide use of a composition comprising a partly-hydrolysed fish gelatin for preparation of a food additive or supplement and/or a medical speciality having the features set out in the appended independent claim.

The present invention also relates to a kit having the features set out in the appended independent claim.

Other preferred embodiments of the present invention are described in the appended sub-claims. Further technical features and the advantages of the invention will be best understood from the following detailed description.

### DESCRIPTION OF THE PREFERRED EMBODIMENTS

The partly-hydrolysed gelatin being the object of the present invention is conceived as an available source of amino acids easily assimilable by the organism. Preferably, the natural fish gelatin is submitted to a hydrolysis treatment. Hydrolysis can be carried out by chemical route or, alternately, by enzymatic route following methods that are known to those skilled in the art. The true hydrolysis is carried out until a partly-hydrolysed gelatin is obtained in which the macromolecules have a molecular weight not exceeding 50,000 Daltons. The degree of partial hydrolysis is obtained in such a manner that the native fish gelatin, following the hydrolysis process, loses its native original structure. The native structure is responsible for gelatin gelation. Hydrolysis causes breaking of the interchain bonds of the native gelatin and does not involve formation of free sulfurised amino acids. Treatment to which collagen (in this case fish collagen) is submitted brings to achievement of a gelatin form which is cold-soluble in water, which has lost its gel-forming feature. The product is defined as a non-gelling water-soluble gelatin. The particular chemical structure of partly-hydrolysed gelatin which is the object of the present invention allows the gelatin to be easily assimilable by the organism and, consequently allows use of lower dosages than those required if native gelatin were used. In addition, since the partly-hydrolysed gelatin being the object of the invention does not contain free sulfurised amino acids, it has no disagreeable aftertaste which is typical of the last-mentioned products. Finally, presently there are not in the literature announcements of a possible allergenic power which was on the contrary practically found and mentioned in the literature with reference to native gelatin.

The partly-hydrolysed gelatin which is the object of the present invention achieves two important advantages with respect to both the fully hydrolysed gelatin and the reconstituted gelatin obtained by mixing the constituent amino acids thereof in their original proportion:

it improves acceptability of treatment by a patient avoiding the disagreeable aftertaste due to free sulfurised amino acids;

it greatly reduces the treatment cost.

As compared with native gelatin, the partly-hydrolysed gelatin on the contrary has the following advantages:

it improves treatment acceptability by the patient avoiding the feeling of gastric swelling due to gelation;

it reduces the required doses for obtaining the desired effects.

This result could be expected in the light of the prior art that led to think that a full hydrolysis of the native gelatin was necessary and essential for improving absorption of the gelatin-constituent amino acids and reduce active dosages thereof.

The partly-hydrolysed gelatin being the object of the present invention applies to all gelatin uses that are not linked to its original physico-chemical properties, such as swelling in the presence of water, but that are linked to its ability to supply the necessary amino acids in a readily absorbable form where there is a lack of. amino acids or under unhealthy conditions that can take advantage of an additional supply of amino acids.

In an embodiment being the object of the invention the partly-hydrolysed fish gelatin is provided for use as a food additive and/or for use as a medical speciality.

Another embodiment being the object of the invention provides a composition comprising a partly-hydrolysed fish gelatin for use as a food additive and/or for use as a medical speciality.

The compositions comprising partly-hydrolysed gelatin efficaciously apply in the medical field and in the veterinary field. In the veterinary field, the compositions comprising partly-hydrolysed gelatin can be used with reference to pets such as dogs and cats for example.

The partly-hydrolysed fish gelatin is used for preparation of food additives for preventive and/or curative treatment of diseases associated with a lack of amino acids in the living organism. In particular, for the preventive treatment of, and/or as an adjuvant in osteoporosis, alopecia, convalescence, senescence, pregnancy, nursing, altered trophism of micro circulation and veins and in some parodontitis.

Preferably, the composition of the food additive and/or medical speciality is in a form adapted for oral administration.

In addition, the compositions being the object of the present invention can be employed as adjuvants in therapies where use of other specific drugs is provided.

Advantageously, the composition of the food additive and/or medical speciality is in the form of a ready-dissolution granular powder, masticable tablets or effervescent tablets. In addition, water-based sterile solutions comprising the compositions of the present invention already in a dissolved form ready for use can be prepared.

A preferred embodiment of the present invention consists of a kit. The Applicant has conceived a new form of package enabling all consumers/patients to have at their disposal and take advantage of a simple, useful and practical amino acid supply means.

It is an object of the present invention to provide a kit comprising: a first container X holding a composition comprising vitamins; and a second container Y holding the partly-hydrolysed fish gelatin.

Alternatively, said first container X may hold a solubilizing liquid, a water-based sterile solution for example, and said second container Y may hold the composition comprising the partly-hydrolysed fish gelatin being the object of the present invention.

Alternatively, said container X may hold a mixture in a preferably anhydrous solid form comprising mineral salts, vitamins, amino acids, flavours, pH controls, co-formulation aids, additives and substances necessary to a correct formulation of the food additive and/or medical speciality and said container Y may hold the partly-hydrolysed fish gelatin.

Preferably, vitamins are selected from the group comprising: vitamin A, vitamin B₁, vitamin B₂, vitamin B₃, vitamin B₆, vitamin C, vitamin D₃, vitamin E and vitamin H.

Contents of said first container X are added to contents of said second container Y, at the moment of use, by the consumer/patient.

Alternatively, said container Y may hold the partly-hydrolysed fish gelatin in a solid form, in the form of a granular powder or micro capsules for example, or it may hold the partly-hydrolysed fish gelatin solubilized in a preferably water-based solubilizing liquid.

Advantageously, the partly-hydrolysed fish gelatin is in combination with calcium carbonate and vitamin D₃ (cholecalciferol).

Advantageously, the composition of the food additive and/or medical speciality efficiently applies under unhealthy conditions, such as senescence, convalescence, pregnancy, and chronic renal insufficiency and in sports activities.

In addition, the compositions of the invention are employed in parodontitis; in particular in tooth diseases connected with both thinning of the bony tissue and weakening of dental ligaments.

The compositions of the invention efficiently apply for the above mentioned cases as an alternative to other amino acid sources, such as meat that can be assimilated less easily or can involve taking of useless or dangerous components.

In addition, they can be employed in association with low-calorie diets in treating obesity in order to avoid the risk of an insufficient amino acid supply.

The compositions of the invention can be administered to patients through true treatment cycles.

For instance, a treatment cycle involves administration of an amount of partly-hydrolysed fish gelatin included from 4 to 10 g/day. Preferably, in an amount of 5-6 g/day.

For instance, administration takes place twice a day at the main meals for a period of time of 8 to 16 weeks. Preferably for a period of time in the range of 10 to 12 weeks. In addition, provision is made for a method involving preventive and/or curative treatment of diseases sharing a deficient supply or an increased consumption of amino acids, or a defect in the amino acid absorption in the organism comprising at least one treatment cycle during which a composition comprising partly-hydrolysed fish gelatin as claimed is administered.

Oral Absorption of the Amino Acids contained in the Gelatin

To two parallel groups of eight rabbits on an empty stomach 100 mg/kg of partly-hydrolysed (non gelling) fish gelatin with an average molecular weight not exceeding 50,000 Dalton and respectively 100 mg/kg of reference gelatin of bovine origin, non hydrolysed and with marked gelling properties at room temperature were administered through a gastric tube. At predetermined times samples of blood from the central artery of the ear were carried out to determine the plasma concentration of the free amino acids. The analysis was conducted after precipitation of the plasma proteins, lyophilizing of the residue, transformation into a derivative by phenyl isothiocyanate and HPLC analysis Pico Tag®. The quantitative determination was carried out on six amino acids present in greater quantities in the gelatin and more particularly: glycine, alanine, arginine, proline, hydroxyproline and glutamic acid. Data reproduced in Table 1 and FIG. 1 are expressed in nmoles/ml as the sum of the six amino acids-analysed after subtraction of basal values. From data in Table 1 and in the graph in FIG. 1, it is possible to infer that absorption of partly-hydrolysed fish gelatin, evaluated in terms of free amino acids present in the blood, is about twice with respect to the reference gelatin. The increase is almost superimposable on that previously reproduced with the fully hydrolysed gelatin. The partly-hydrolysed gelatin can be used as a base for preparations adapted for treatment of different pathologic conditions.

In addition the partly-hydrolysed gelatin can be administered in formulations of different form such as ready-dissolution granular powder, masticable pills, tablets soluble in mouth, effervescent tablets, masticable tablets and kit. By way of non-limiting example of the object of the present invention, some examples of compositions comprising partly-hydrolysed fish gelatin are reproduced hereinafter. The compositions of the following examples were prepared following knowledge and techniques known to a person skilled in the art.

### EXAMPLES

### Example 1

Ready-dissolution granular powder to be used in osteoporosis (1 packet of granular powder contains):

| | | |
|---|---|---|
| Partly-hydrolysed fish gelatin | g | 2.50 |
| Calcium carbonate (with addition of | g | 1.10 |
| maltodextrin) | | |
| Vitamin D₃ (cholecalciferol) | μg | 2.50 |
| Sodium benzosulfimide | mg | 50 |
| Strawberry/vanilla flavour | mg | 114 |
| Citric acid | g | 1.76 |
| Mannitol | mg | 250 |
| 50% dimethicone powder | mg | 60 |
| Red colour | mg | 2 |

### Example 2

Tablet

| | | |
|---|---|---|
| Partly-hydrolysed fish gelatin | g | 2.50 |
| Calcium carbonate (with addition of | g | 1.10 |
| maltodextrin) | | |
| Vitamin D₃ (cholecalciferol) | μg | 2.50 |
| Sodium benzosulfimide | mg | 50 |
| Strawberry/vanilla flavour | mg | 117 |
| Citric acid | g | 1.50 |
| Mannitol | mg | 250 |
| 50% dimethicone powder | mg | 60 |
| PEG 6000 | mg | 60 |
| Red colour | mg | 2 |

### Example 3

Ready-dissolution granular powder to be used in convalescence (1 packet of granular powder contains):

| | | |
|---|---|---|
| Partly-hydrolysed fish gelatin | g | 2.50 |
| Lysine hydrochloride | mg | 150 |
| Taurine | mg | 50 |
| Vitamin B₆ | mg | 1 |
| Vitamin C | mg | 45 |
| Sodium benzosulfimide | mg | 50 |
| Sodium cyclamate | mg | 50 |
| Banana flavour | mg | 154 |

### Example 4

Ready-dissolution granular powder to be used in senescence (1 packet of granular powder contains):

| | | |
|---|---|---|
| Partly-hydrolysed fish gelatin | g | 2.50 |
| EPA- and DHA-rich sea fish oil | g | 2 |
| Vitamin E | mg | 10 |
| Vitamin C | mg | 45 |
| Betacarotene | mg | 5 |
| Sodium benzosulfimide | mg | 50 |
| Sodium cyclamate | mg | 50 |
| Vanilla flavour | mg | 150 |

### Example 5

Ready-dissolution granular powder to be used in pregnancy and/or nursing (1 packet of granular powder contains):

| | | |
|---|---|---|
| Partly-hydrolysed fish gelatin | g | 2.50 |
| Lysine hydrochloride | mg | 150 |
| Iron gluconate (corresponding to | mg | 60 |
| mg 7 iron) | | |
| Calcium glycerophosphate | g | 1 |
| Vitamin A | mg | 0.4 |
| Vitamin B₁ | mg | 0.5 |
| Vitamin B₂ | mg | 0.5 |
| Vitamin B₃ | mg | 7.5 |
| Vitamin B₆ | mg | 1 |
| Vitamin C | mg | 25 |
| Vitamin D₃ (cholecalciferol) | μg | 2.5 |
| Vitamin H | mg | 0.07 |
| Sodium benzosulfimide | mg | 50 |
| Vanilla flavour | mg | 150 |

### Example 6

Ready-dissolution granular powder to be used in micro circulation and vein troubles (1 packet of granular powder contains):

| | | |
|---|---|---|
| Partly-hydrolysed fish gelatin | g | 2.50 |
| Centella Asiatica (triterpene fraction) | mg | 15 |
| Vitamin C | mg | 45 |
| Vitamin E | mg | 7 |
| Sodium benzosulfimide | mg | 50 |
| Sodium cyclamate | mg | 50 |
| Strawberry flavour | mg | 140 |

### Example 7

Ready-dissolution granular powder to be used in association with low-calory diets (1 packet of granular powder contains):

| | | |
|---|---|---|
| Partly-hydrolysed fish gelatin | g | 2.50 |
| Sodium benzosulfimide | mg | 50 |
| Strawberry flavour | mg | 140 |

### Example 8

Ready-dissolution granular powder to be used as medical speciality in osteoporosis (1 packet of granular powder contains):

| | | |
|---|---|---|
| Partly-hydrolysed fish gelatin | g | 2.50 |
| Calcium carbonate (with addition of | g | 1.25 |
| maltodextrin) | | |
| Vitamin D₃ (cholecalciferol) | μg | 4.4 |
| Sodium benzosulfimide | mg | 50 |
| Strawberry/vanilla flavour | mg | 114 |
| Citric acid | g | 2.175 |
| Mannitol | mg | 250 |
| 50% dimethicone powder | mg | 60 |
| Red colour | mg | 2 |

TABLE 1

| | | | Reference gelatin | | | Fully-hydrolysed bovine gelatin | | |
|---|---|---|---|---|---|---|---|---|
| Partly-hydrolysed fish gelatin | | | | | Stan- | | | Stan- |
| Time | nmol/ | Stan- | Time | nmol/ | dard | Time | nmol/ | dard |
| (hr) | mL | dard error | (hr) | mL | error | (hr) | mL | error |
| 0 | 0 | 0 | 0 | 0 | 0 | 0 | 0 | 0 |
| 0.5 | 216.01 | 41.74 | 0.5 | 131.50 | 28.43 | 0.5 | 285.10 | 64.22 |
| 1 | 405.75 | 70.44 | 1 | 190.13 | 34.39 | 1 | 424.74 | 84.16 |
| 2 | 183.13 | 45.51 | 2 | 81.38 | 16.69 | 2 | 193.21 | 41.89 |
| 3 | 85.88 | 36.21 | 3 | 5.25 | 17.74 | 3 | 91.40 | 49.58 |
| 4 | 31.50 | 30.45 | 4 | 10.50 | 25.29 | 4 | 33.84 | 62.81 |
| 6 | 10.63 | 22.86 | 6 | −49.75 | 32.85 | 6 | 11.98 | 70.18 |

[image]

What is claimed is:

1. A partly-hydrolysed fish gelatin, for use as a food additive, wherein said gelatin is water-soluble and has a molecular weight not exceeding 50,000 Daltons.

2. The gelatin as claimed in claim 1, wherein said gelatin is substantially devoid of inter-chain bonds between the proteinic polymer chains constituting it and does not possess a gelling property.

3. The gelatin as-claimed in claim 2, wherein said gelatin does not contain sulfurised amino acids in a free form.

4. A method for treatment of a condition of a patient, wherein the treatment involves administering to the patient an amount of partly-hydrolysed fish gelatin, said fish gelatin being water-soluble and having a molecular weight not exceeding 50,000 Daltons, said condition being selected from the group consisting of (a) a deficient supply of amino acids or a defective absorption of amino acids in said patient, (b) osteoporosis, alopecia or trophism of the micro circulation and veins in said patient, (c) parodontitism in said patient, and (d) tooth diseases connected with both thinning of the bony tissue and weakening of dental ligaments in said patient.

5. The method of claim 4, wherein said condition is a deficient supply of amino acids or a defective absorption of amino acids in said patient.

6. The method of claim 4, wherein said condition is osteoporosis, alopecia or trophism of the micro circulation and veins in said patient.

7. The method of claim 4, wherein said condition is parodontitism in said patient.

8. The method of claim 4, wherein said condition is tooth diseases connected with both thinning of the bony tissue and weakening of dental ligaments in said patient.

9. A composition, for use as a food additive or medicament, comprising partly-hydrolysed fish gelatin as claimed in claim 1 and at least one vitamin selected from the group consisting of: vitamin A, vitamin B₁, vitamin B₂, vitamin B₃, vitamin B₆, vitamin C, vitamin D₃, vitamin E and vitamin H.

10. The composition as claimed in claim 9, in a form adapted for oral administration.

11. The composition as claimed in claim 9, in the form of a granular powder, masticable tablets and effervescent tablets.

12. A method for treatment of a condition of a patient, wherein the treatment involves administering to the patient an amount of a composition, said composition comprising partly-hydrolysed fish gelatin and at least one vitamin selected from the group consisting of: vitamin A, vitamin B₁, vitamin B₂, vitamin B₃, vitamin B₆, vitamin C, vitamin D₃, vitamin E and vitamin H, said fish gelatin being water-soluble and having a molecular weight not exceeding 50,000 Daltons, said condition being selected from the group consisting of (a) a deficient supply of amino acids or a defective absorption of amino acids in said patient, (b) osteoporosis, alopecia or trophism of the micro circulation and veins in said patient, (c) parodontitism in said patient, and (d) tooth diseases connected with both thinning of the bony tissue and weakening of dental ligaments in said patient.

13. The method of claim 12, wherein said condition is a deficient supply of amino acids or a defective absorption of amino acids in said patient.

14. The method of claim 12, wherein said condition is osteoporosis, alopecia or trophism of the micro circulation and veins in said patient.

15. The method of claim 12, wherein said condition is parodontitism in said patient.

16. The method of claim 12, wherein said condition is tooth diseases connected with both thinning of the bony tissue and weakening of dental ligaments in said patient.

17. A kit comprising:
a first container X holding a composition comprising vitamins, and
a second container Y holding the partly-hydrolysed fish gelatin, as claimed claim 1; both said containers being closed and said container X being disposed on container Y so as to join the contents of said container X into said container Y, at the moment of use.
